(19) United States
(12) Patent Application Publication
(10) Pub. No.: US 2025/0295834 A1
(43) Pub. Date: Sep. 25, 2025

(54) SUBTISSUE IMPLANT MATERIAL

(71) Applicant: Cook Biotech Incorporated, West Lafayette, IN (US)

(72) Inventors: Sydney E. Hollingshead, Lafayette, IN (US); Bhavin B. Shah, West Lafayette, IN (US)

(73) Assignee: Cook Biotech Incorporated, West Lafayette, IN (US)

(21) Appl. No.: 18/863,067

(22) PCT Filed: May 5, 2023
(86) PCT No.: PCT/US2023/066683

§ 371 Date: Nov. 5, 2024

Related U.S. Application Data
(60) Provisional application No. 63/338,656, filed on May 5, 2022.
(60) Provisional application No. 63/450,755, filed on Mar. 8, 2023.

(51) Int. Cl. A61L 27/22; A61L 27/36

(52) U.S. Cl. CPC A61L 27/222; A61L 27/3604; A61L 2400/06

(57) ABSTRACT

In certain embodiments, the present disclosure provides methods of treating diseased or damaged tissue in a patient. In some forms the methods comprise introducing a photocurable composition to the diseased or damaged tissue, the photocurable composition comprising a photoactivatable metal ligand complex, a matrix forming polymer, and an electron acceptor. Such methods may further comprise irradiating the photocurable composition through a tissue portion to induce a photocrosslinking reaction forming crosslinks between individual polymers of the matrix forming polymer within the photocurable composition.

### CROSS REFERENCE TO RELATED APPLICATION

This application claims the benefit of U.S. Provisional Application No. 63/338,656 filed May 5, 2022, and U.S. Provisional Application 63/450,755 filed Mar. 8, 2023, which are hereby incorporated herein by reference in their entirety.

### BACKGROUND

The present invention resides generally in the field of medical compositions and in particular aspects to medical compositions that incorporate extracellular matrix materials.

As further background, subdermal injections of various materials are used to fill defects or voids in soft tissue during reconstructive surgery after serious disease and traumatic injuries. Some patients may have disease that cause soft tissue to irreversibly degrade or waste away, creating physical and psychological effects. In addition, severe injuries may remove significant portions of soft tissue, and other infectious diseases may require the removal of significant amounts of soft tissue during surgery. In situations where small portions of soft tissue are lost, the patient may be treated with injections of a natural or synthetic polymer solution. These solutions are usually water-based and high in viscosity to increase biocompatibility and pliability in the soft tissue. Some may be very lightly gelled solutions of collagen. Some treatments may use fat tissue autografts from elsewhere in the patient's body. Tn situations where large portions of soft tissue are lost, the treatment usually involves covering the soft tissue void with a skin graft and allowing the body to regrow as much tissue as it can. These patients often never recover all the lost soft tissue (including muscle, fat, etc.) and experience physical and psychological effects of this absence of tissue. Some of these treatments also see overlap into the cosmetic surgery space, where subdermal injections are used to reduce the appearance of wrinkles, acne and other scars, hollow areas, and generally increase the fullness of the face.

Because these injected filler solutions are liquid and degradable, they inherently degrade relatively quickly after implantation. For example, hyaluronic acid and autograft injections last from three to twelve months, while longer lasting treatments like calcium hydroxyapatite lasts from twelve to twenty-four months.

A need remains for additional medical compositions and products that can be used in a wide variety of medical applications, in particular to fill a soft tissue void with a lasting and durable material. The present disclosure provides such medical compositions and products, as well as methods for preparing and using the same.

### SUMMARY

In certain aspects, the present disclosure pertains to unique systems and methods for treating diseased or damaged tissue in a patient. In accordance with some forms of the disclosure, such methods including introducing a photocurable composition to the diseased or damaged tissue. Accordingly, in one embodiment, the present disclosure provides a method of treating diseased or damaged tissue in a patient, the method comprising introducing a photocurable composition to the diseased or damaged tissue, the photocurable composition comprising a photoactivatable metal ligand complex, a matrix forming polymer, and an electron acceptor such that the photocurable composition is positioned behind a tissue portion. In some forms the disclosed methods comprise irradiating the photocurable composition through the tissue portion to induce a photocrosslinking reaction forming crosslinks between individual polymers of the matrix forming polymer within the photocurable composition. In accordance with certain inventive variants, introducing comprises injecting the photocurable composition into the patient. In certain embodiments the tissue portion has a thickness of about 1 mm to about 50 mm. In some forms, the irradiating step is performed with an external light source. In accordance with some forms the external light source is configured to emit visible light.

In another embodiment, the disclosure provides a method of treating a depressed tissue portion in a patient, the method comprising injecting a photocurable composition beneath the depressed tissue portion, the photocurable composition comprising a photoactivatable metal ligand complex, a matrix forming polymer, and an electron acceptor. In some forms the disclosed methods comprise irradiating the photocurable composition through the patient's skin to induce a photocrosslinking reaction forming crosslinks between individual polymers of the matrix forming polymer within the photocurable composition. In accordance with certain inventive variants, introducing comprises injecting the photocurable composition into the patient. In certain embodiments the tissue portion has a thickness of about 1 mm to about 50 mm. In some forms, the irradiating step is performed with an external light source. In accordance with some forms the external light source is configured to emit visible light.

In another embodiment, the disclosure provides a medical kit comprising a sterile package, a photocurable composition contained within the sterile package, the photocurable composition comprising a photoactivatable metal ligand complex, a matrix forming polymer, and an electron acceptor, and a syringe configured to inject the photocurable composition into a subdermal or intramuscular location of a patient. In accordance with some forms, the medical kit also comprises comprising a light source configured to induce a photocrosslinking reaction within the photocurable composition upon exposure to irradiation from the light source through patient tissue.

Additional embodiments, as well as features and advantages of embodiments of the invention, will be apparent from the description herein.

### BRIEF DESCRIPTION OF THE DRAWINGS

FIGS. 1A-1D illustrate one embodiment of a method of filing a soft tissue void.

FIG. 2 illustrates a tissue section including a depressed tissue portion.

FIGS. 3A and 3B illustrate one embodiment of a method of treating a depressed tissue portion.

FIG. 4 illustrates one embodiment of a photocurable composition introduced into a subcutaneous tissue location.

FIG. 5 illustrates one embodiment of a photocurable composition introduced into a intramuscular tissue location.

FIG. 6 illustrates one embodiment of a photocurable composition introduced into a intradermal tissue location.

FIG. 7 illustrates the results of the experiment detailed in Example 3, detailing the extent of modification with varying Bolton Hunter reagent concentration.

### DESCRIPTION OF THE SELECTED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated graft, and further applications of the principles of the disclosure as illustrated therein are herein contemplated as would normally occur to one skilled in the art to which the disclosure relates.

As disclosed above, aspects of the present disclosure relate to novel methods and materials for treating diseased and damaged tissues. Such methods and materials may be useful, for example to fill a tissue void without invasive surgical intervention. In certain aspects, the disclosure relates to introducing a photocurable composition as described herein to a location under the skin of a patient, or covered by a tissue graft, and irradiating the photocurable composition through the patient skin or tissue graft to cause a photocrosslinking reaction within the photocurable composition. In some forms such a method is performed to bulk sunken or otherwise damaged tissue such as scar tissues. Thus, the present disclosure provides methods for in situ polymerization to provide lasting cosmetic and/or medical corrective procedures. In general, the photocurable compositions of the present disclosure are introduced under the skin, or tissue graft, and polymerized by exposure to visible light applied to the skin surface, from outside of the patient's body.

Thus, in accordance with some forms, the present disclosure provides a photocurable composition comprising a photoactivatable metal ligand complex, a matrix forming polymer, and an electron acceptor. In certain aspects, the present disclosure relates to a photocurable compositions that include a polymer containing phenolic groups, such photocurable compositions are effective to form a di phenolic crosslinked polymer hydrogel when photocured. While not wishing to be bound by theory, it is believed that the photocrosslinking mechanism involves irradiation of the metal-ligand complex to induce an excited state, followed by transfer of an electron from the metal to the electron acceptor. The oxidized metal then extracts an electron from a side chain in the matrix forming polymer. In certain embodiments the side chain is a tyrosine side chain and the reaction produces a tyrosyl radical that reacts immediately with a nearby tyrosine to form a dityrosine bond. Thus, in accordance with some forms of practicing the disclosed methods, a direct cross-link (without any bridging moiety) is created quickly in this photo-initiated chemical reaction, without the need for introduction of a primer layer and without the generation of potentially detrimental species such as singlet oxygen, superoxide and hydroxyl radicals. It has been discovered that a cross-linking reaction may occur in the absence of a photoactivatable metal-ligand complex. Such formulations require extended curing time, for example at least two hours, and potentially up to about 24 hours. In this way, compositions of the present disclosure, with or without a metal ligand complex, may form crosslinks in the absence of light. Thus, the methods disclosed herein may be practiced without irradiating the injected composition with light, such methods require a curing time of at least two hours, and may not be fully crosslinked for about 24 hours.

As used herein, the term “photoactivatable metal-ligand complex” means a metal-ligand complex in which the metal can enter an excited state when irradiated such that it can donate an electron to an electron acceptor in order to move to a higher oxidation state and thereafter extract an electron from a side chain of an amino acid residue of a matrix polymer to produce a free radical without reliance upon the formation of singlet oxygen. Suitable metals include but are not limited to Ru(II), Pd(I), Cu(II), Ni(II), Mn(II) and Fe(III) in the form of a complex which can absorb light in the visible region, for example, an Ru(II) bipyridyl complex, a Pd(II) porphyrin complex, a sulfonatophenyl Mn(I) complex or a Fe(I) protoporphyrin complex, more particularly, an Ru(II) bipyridyl complex or a Pd(II) porphyrin, in particular, an Ru(III) (bpy)₃ complex such as [Ru(II) (bpy)b]Cl₂. Efficient cross-linking occurs in the presence of an electron acceptor, and requires only moderate intensity visible light.

As used herein the term “electron acceptor” refers to a chemical entity that accepts an electron transferred to it and so refers to an easily reduced molecule (or oxidizing agent) with a redox potential sufficiently positive to facilitate the cross-linking reaction. A range of electron acceptors will be suitable. In an embodiment, the electron acceptor is a peracid, a cobalt complex, a cerium (IV) complex, or an organic acid. Typically, the electron acceptor is a persulfate, periodate, perbromate or perchlorate compound, vitamin B12, Co(III) (NH₃)SCI²⁻ cerium (IV) sulphate dehydrate, ammonium cerium (TV) nitrate, oxalic acid or EDTA. Preferably, the persulfate anion is used as the electron acceptor. In accordance with certain embodiments, the electron acceptor comprises sodium persulfate. The standard oxidation-reduction potential for the reaction:

S₂O₈²⁻+2H⁻+2e⁻=>2 HSO₄₋

is 2.1 V, as compared to 1.8 V for hydrogen peroxide (H₂O₂). This potential is higher than the redox potential for the permanganate anion (MnO₄₋) at 1.7 V, but slightly lower than that of ozone at 2.2 V.

As used herein the term “matrix forming polymer” refers to isolated and purified extracellular matrix proteins as well as synthetic polymers. Suitable matrix proteins for use in the coating may be selected from, but not limited to the group consisting of: fibrinogen, fibrin, collagen, keratin, gelatin, fibronectin, serum albumin, elastin, beta-lactoglobulin, glycinin, glutens, gliadins, resilin and/or laminin, or admixtures thereof. Matrix proteins may be isolated from human or animal sources or can be synthetically produced for instance using recombinant techniques. In some forms, matrix proteins are isolated from ECM source tissues as described herein. In some forms, the protein may be denatured to encourage the formation of phenolic cross-links upon photocuring and/or may be a phenol enriched protein (e.g. modified to increase the number of tyrosine groups). Denaturation of a protein may be accomplished by raising or lowering the pH of a solution containing the matrix protein, decreasing or increasing the ionic strength of a solution containing the matrix protein, hydrolysis, or in other ways known to a person skilled in the art. Chemical modification to form a phenol enriched polymer material (e.g. including chemically added tyrosine groups) may be achieved by any suitable method. In some forms, such chemical modification may include the modification of amino acid side chains to include aromatic moieties such as the phenolic moiety present in tyrosine. By way of example primary amines such as the lysine residues in a protein may be modified using the known Bolton Hunter reagent (N-succinimidyl-3-[4-hydroxyphenyl]propionate) or the known water-soluble Bolton Hunter reagent (sulfosuccinimidyl-3-[4-hydroxyphenyl]propionate). In certain forms, the photocurable adhesive will include a mixture of an amount of a protein (especially collagen, gelatin or a collagen peptide composition) with an amount of the corresponding phenol enriched protein. For example, the photocurable adhesive may include the parent (unmodified) protein and the corresponding phenol enriched protein in a dry weight ratio in the range of about 1:10 to about 10:1, or about 1:5 to about 5:1, and in some forms about 5:1 to about 2:1. In certain embodiments, such ratios are used when the parent (unmodified) protein is collagen, gelatin, or a collagen peptide composition.

It is also within the scope of the present disclosure to include one or more synthetic polymers in the matrix forming polymer. Exemplary synthetic polymers include phenol-containing polymers, such as polyacrylamide and/or polyacrylic acid. Alternative embodiments may include a biodegradable polymer additive such as one or more of: polyethylene glycol, polyvinyl alcohol, polylactic acid, polyglycolic acid, polylactic-co-glycolic acid, and/or polyglycerol sebacate. In certain embodiments, the polymer is phenol-enriched by using Bolton-Hunter reagent or other suitable reaction.

The term “phenol enriched” as applied to a polymer material herein (e.g. collagen, gelatin, a collagen peptide composition, or synthetic polymer) means that the polymer material has been chemically modified to increase the number of phenolic groups in the polymer material. Thus, “phenol enriched collagen” refers to collagen that has been chemically modified to increase the number of phenolic groups (e.g. tyrosine groups) in the collagen, “phenol enriched gelatin” refers to gelatin that has been chemically modified to increase the number of phenolic groups in the gelatin, and “phenol enriched collagen peptide composition” refers to a collagen peptide composition that has been chemically modified to increase the number of phenolic groups in the collagen peptide composition. In some aspects, the phenolic groups are tyrosine groups, which can be added for example using a known Bolton Hunter reagent. In some aspects, the phenol enriched polymer material (e.g. collagen, gelatin, or collagen peptide composition) will have a P/G value of at least about 7, and in certain forms in the range of about 7 to about 35, or in the range of about 15 to about 30, or in the range of about 18 to about 25, where the P/G value is the number of moles of phenol groups per mole of polymer in the polymer material. The P/G value for a polymer material can be determined using standard techniques, including for example using an absorbance assay at a wavelength of 280 nm. Moderate P/G ranges for the phenol enriched polymer, as recited above, are preferred in some aspects, as modification to higher P/G values has been found to decrease the solubility of the material in aqueous media (see e.g. Example 3 below for phenol enriched gelatin).

In certain embodiments a photocurable composition as described herein may be injected beneath the surface of the skin. In some forms the photocurable composition is injected within, proximal to, or beneath diseased or damaged tissue and cured to bulk the depressed tissue portion. Such diseased or damaged tissues may be scarred or otherwise sunken tissues. In certain embodiments the photocurable compositions of the present disclosure may be injected within laryngeal tissue, for example to modify or support the vocal folds. In some forms the photocurable composition is used to treat, for example lesions, polyps, nodules, and/or cysts on the vocal fold and/or voids left after removal of vocal fold lesions, polyps, nodules, and cysts, scarred vocal folds, and/or paralysis of vocal folds. Compositions of the present disclosure may be used in a method for treating vocal fold paralysis, for example unilateral vocal fold paralysis. Such methods may include injecting a photocurable composition as described herein into the vocal fold and irradiating the photocurable composition through the vocal fold tissue so as to move the vocal fold in a more medial direction to facilitate contact with the opposing vocal fold. Compositions of the present disclosure may be used in a method for treating lesions, polyps, nodules, and/or cysts on the vocal fold. Such methods may include injecting a photocurable composition as described herein into the affected vocal fold tissue (e.g. directly into or adjacent to the lesion, polyp, nodule, and/or cyst) and thereafter irradiating the photocurable composition through the tissue. Compositions of the present disclosure may be used in a method for treating voids left after removal of vocal fold lesions, polyps, nodules, and/or cysts. Such methods may include injecting a photocurable composition as described herein into patient tissue near such a void (e.g. underlying or adjacent to the void) and thereafter irradiating the photocurable composition through the tissue. In certain embodiments, the photocurable compositions of the present disclosure may be injected within or near sphincter tissue, for example to modify or support the anal sphincter or the urethral sphincter. In some forms the photocurable composition is used to treat, for example fecal incontinence, and/or urinary incontinence. In this way the photocurable composition is injected into or near the sphincter tissue beneath the surface of the skin, or other superficial tissue layer, such that the photocurable composition is positioned behind (e.g. deep to) a tissue portion. In accordance with certain embodiments the tissue portion has a thickness of about 0.5 mm to about 100 mm, preferably about 1 mm to about 50 mm. Put another way the photocurable compositions as disclosed herein may be injected to a location having a depth beneath the surface of the skin of between about 0.5 mm to about 100 mm, preferably about 1 mm to about 50 mm.

In accordance with some forms, the photocurable composition is provided as a separate photocurable composition precursor and an activator. Methods of the present disclosure may comprise mixing a photocurable composition precursor and activator to form the photocurable composition. In certain embodiments, the photocurable composition precursor comprises a photoactivatable metal ligand complex and a matrix forming polymer as described herein. In certain embodiments, the activator comprises an electron acceptor. In certain preferred forms, the photocurable composition precursor is comprised of an aqueous medium that includes a polymer, or two or more polymers containing phenolic groups and a photoactivatable metal-ligand complex. In some forms, the separate components, the photocurable composition precursor and activator, may be provided in separate syringes. In certain embodiments a luer to luer connector is provided to enable combining of the photocurable composition precursor and activator prior to use to form an aqueous photocurable composition. In other embodiments a double barrel mixing syringe, or two separate syringes or vials, may be supplied having the photocurable composition precursor in a first chamber and the activator in a second chamber.

In some forms, the photocurable composition precursor is provided as a sterile liquid preparation in the first chamber including collagen, phenol enriched collagen, gelatin, phenol enriched gelatin, a collagen peptide composition, or a phenol enriched collagen peptide composition. These polymer materials can be used either singly or in combination. For example, the photocurable composition may include a combination of collagen and phenol enriched collagen, a combination of gelatin and phenol enriched gelatin, or a combination of a collagen peptide composition and a phenol enriched collagen peptide composition. In each case, the dry weight ratio of the parent polymeric material and its phenol enriched counterpart can be in the range of about 1:10 to about 10:1, or about 1:5 to about 5:1, or in some forms about 1:5 to about 1:2. Mixtures of two or more of collagen, gelatin, and a collagen peptide composition (each in its native form without phenol enrichment or as a phenol enriched polymeric material) can also be used.

In addition or alternatively, the sterile liquid preparation that includes collagen, phenol enriched collagen, gelatin, phenol enriched gelatin, a collagen peptide composition, or a phenol enriched collagen peptide composition, or any mixture of two or more thereof, can exhibit the property of not gelling at 20° C., for example exhibiting no thermoreversible gelation activity upon cooling, or having a thermoreversible gelation temperature below 20° C., or below 15° C. In some forms, the sterile liquid preparation comprises gelatin, phenol enriched gelatin, or a mixture thereof, and the liquid preparation also includes an agent that inhibits the thermoreversible gelling of the gelatin (when present) and of the phenol enriched gelatin (when present). Urea is a preferred agent that inhibits this thermoreversible gelling, and can be used for example at a concentration in the range of about 1 molar to 5 molar in the liquid preparation, more typically about 3 molar to about 4.5 molar, and in some forms about 3.8 molar to about 4.5 molar. In other forms, the sterile liquid preparation includes a collagen peptide composition and/or a phenol enriched collagen peptide composition, that has an average molecular weight (M_{w}) below about 20,000 kilodaltons, more preferably below about 15,000 kilodaltons, and typically in the range of about 2,000 to about 12,000 kilodaltons. In these forms, the collagen peptide composition can exhibit no thermoreversible gelation activity upon cooling to 20° C. (or in some typical forms at any temperature), allowing the liquid preparation to remain a liquid at a temperature of 20° C., or at a temperature of 15° C. It will be understood that the liquid preparation may also remain a liquid at temperatures below these specified temperatures, and in general may remain a liquid throughout a temperature range expected to encompass room temperature storage and normal use temperatures, for example in the range of about 20° C. to about 37° C.

The sterile liquid preparation can include the polymer(s) containing phenolic groups in any suitable concentration. In some forms, the total concentration of the polymer(s) present in the sterile liquid preparation will be in the range of about 1% to about 40 weight/volume, more typically about 10% to about 40% weight/volume. In certain preferred forms, the sterile liquid preparation will include collagen, phenol enriched collagen, gelatin, phenol enriched gelatin, a collagen peptide composition, a phenol enriched collagen peptide composition, or any combination thereof, at a concentration in the range of about 20% to about 35% weight/volume, or in the range of about 25% to about 35% weight/volume. In such forms, the sterile liquid preparation, and photocurable composition prepared using it, can be a flowable viscous liquid, for example having a viscosity at 20° C. of greater than about 300 centipoise, or greater than about 500 centipoise, and typically in the range of about 500 to about 20000 centipoise or in the range of about 1000 to about 10000 centipoise.

The sterile liquid preparation can include the metal ligand complex in a suitable amount to catalyze the formation of covalent crosslinks in the formation of the covalently crosslinked hydrogel by photocuring. Where a Ru(II) (bpy)₃ complex such as [Ru(II)(bpy)₃]Cl₂ is used as the metal ligand complex, preferred sterile liquid preparations will include it at a concentration in the range of about 0.2 to about 2 mM, more desirably about 0.4 to about 1 mM. Where the electron acceptor to be mixed with the sterile liquid preparation is in dry powder form, the prepared photocurable composition will have these same concentrations of the metal ligand complex. Where the electron acceptor is provided in a solution to be combined with the sterile liquid preparation, the concentration of the metal ligand complex in the prepared photocurable composition will be reduced relative to that in the sterile liquid preparation. In some such forms, the volume of the sterile liquid preparation, the volume of the solution of electron acceptor, and the concentration of the metal ligand complex in the sterile liquid preparation, can be selected to provide a concentration of the metal ligand complex in the prepared photocurable composition that is within the above-referenced concentration range values given for the sterile liquid preparation.

The sterile liquid preparation can have been terminally sterilized within the first chamber to render the liquid preparation sterile (e.g. using sterilizing radiation applied to a package containing the first container), but in some preferred forms the liquid preparation is sterilely prepared, for example including passage of the liquid preparation through a sterile filter, and then filled into the first chamber in a sterile filling operation. Such sterilely-filled liquid preparations in the first chamber can therefore be free from exposure to sterilizing radiation, and thus can be free from any degradation of the polymer(s) containing phenol groups caused by the sterilizing radiation. In some forms, the liquid preparation can be in a heated condition to reduce its viscosity during passage through the sterile filter. Also, in some forms, the first container having the first chamber containing the sterilely-filled liquid preparation can be sealed within a sterile barrier package under sterile conditions. Further, such sterile barrier package is preferably impermeable to visible light, as can be provided for example by a foil pouch package.

The present disclosure also provides methods of treating a patient comprising applying a tissue graft to diseased or damaged patient tissue, for example an open cutaneous wound. In accordance with some forms, such methods may include applying a photocurable composition as described herein to a wound bed or otherwise damaged tissues, applying a tissue graft to the wound bed, and irradiating the photocurable composition through the tissue graft to induce a photocrosslinking reaction to form a diphenolic crosslinked polymer hydrogel. Tissue grafts may be any suitable graft material, for example extracellular matrix materials as described herein. In accordance with some forms, the tissue grafts have a maximum thickness of between about 0.5 mm to about 100 mm, preferably about 1 mm to about 50 mm. In certain embodiments, upon irradiation crosslinks are formed between polymers of the matrix forming polymer within the photocurable composition and the surrounding patient tissue and/or tissue graft material.

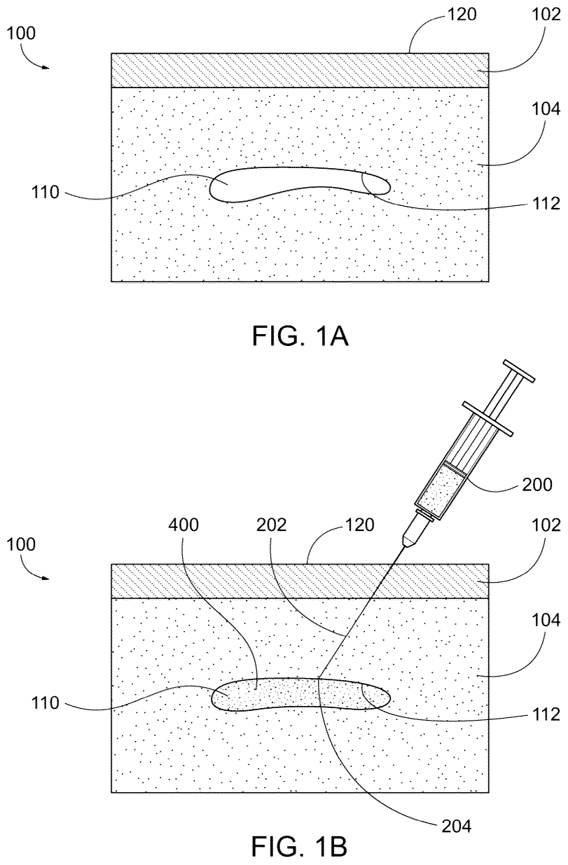

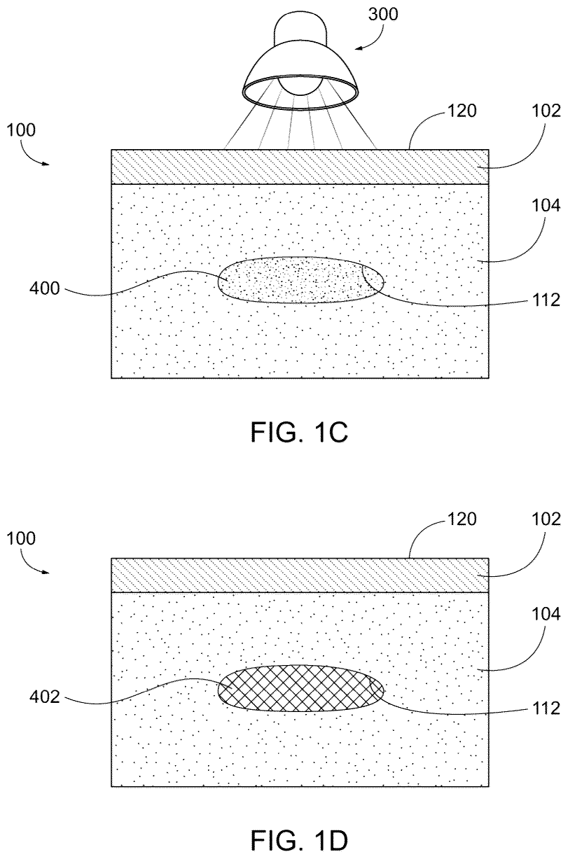

With reference to FIGS. 1A-1D, shown is one embodiment of a method of filling a soft tissue void. Tissue section 100 comprises external tissue layer 102 having external surface 120, and internal tissue layer 104. While shown as two distinct layers it is within the scope of the disclosure to provide a method of filling a soft tissue void present in the external most tissue layer, for example within the epidermis. In the illustrated embodiment, soft tissue void 110 is shown within internal tissue layer, and defined by void walls 112 comprising portions of internal tissue layer 104. Internal tissue layer may comprise one or more soft tissue such as, viscera, muscle, subcutaneous tissue layers, dermis tissue layers, and/or inner layers of the epidermis tissue.

In accordance with some forms of practicing the disclosed methods, a syringe 200 is provided having a needle 202. In certain embodiments, needle 202 is inserted such that needle tip 204 is placed within soft tissue void 110. In the illustrated embodiment needle 202 comprises a cannulated needle having a port at or near needle tip 204, a person having ordinary skill in the art will understand that any suitable delivery means may be used to deliver the photocurable composition, for example a needle having two or more side ports. Thus, in accordance with certain embodiments, photocurable composition 400 is introduced into the soft tissue void 110 through needle 202. As discussed above, the photocurable composition may be premixed and loaded into a single barrel syringe. It is also within the scope of the present disclosure to provide a mixing syringe having two or more chambers containing components of the photocurable solution and which is configured to mix the components prior to, or concurrent with, injecting. Photocurable composition 400 is introduced into soft tissue void 110, for example as shown in FIG. 1B.

In certain embodiments an external light source 300 is used to irradiate the photocurable composition 400. In some forms the external light source is applied externally such that the photocurable composition is irradiated through one or more patient tissue layers existing between the injected or otherwise deposited photocurable composition and the external light source. As discussed elsewhere herein, such irradiation is sufficient to induce a photocrosslinking reaction within the photocurable composition forming crosslinks between individual polymers of the matrix forming polymer within the photocurable composition.

In accordance with some forms, the disclosure provides methods of treating depressed tissues, such as atrophic scars, which cause the depressed portion to sit below the surrounding tissue. Methods of the present disclosure may be employed to bulk or raise depressed tissues.

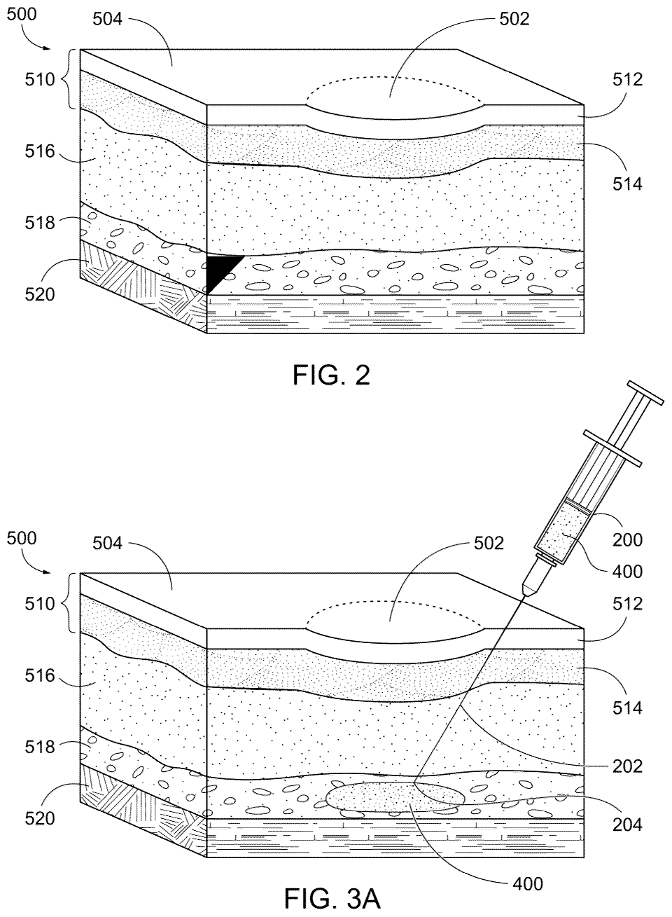

In accordance with certain embodiments the methods disclosed herein may be useful for treating depressed scars resulting from acne. FIG. 2 illustrates a tissue section 500 including depressed tissue portion 502, and skin surface 504. The illustrated example includes various tissue layers including, epidermis tissue layer 510, comprising both superficial epidermal tissue 512 and subsurface epidermal issue 514, dermis tissue layer 516, subcutaneous tissue layer 518, and muscle tissue layer 520. As used herein the term “superficial epidermal tissue” refers to the outermost tissue layer, e.g. the stratum corneum. As used herein the term “subsurface epidermal tissue” refers to epidermal layers beneath the surface of the skin, for example the granular layer, spinous layer, and basal layer.

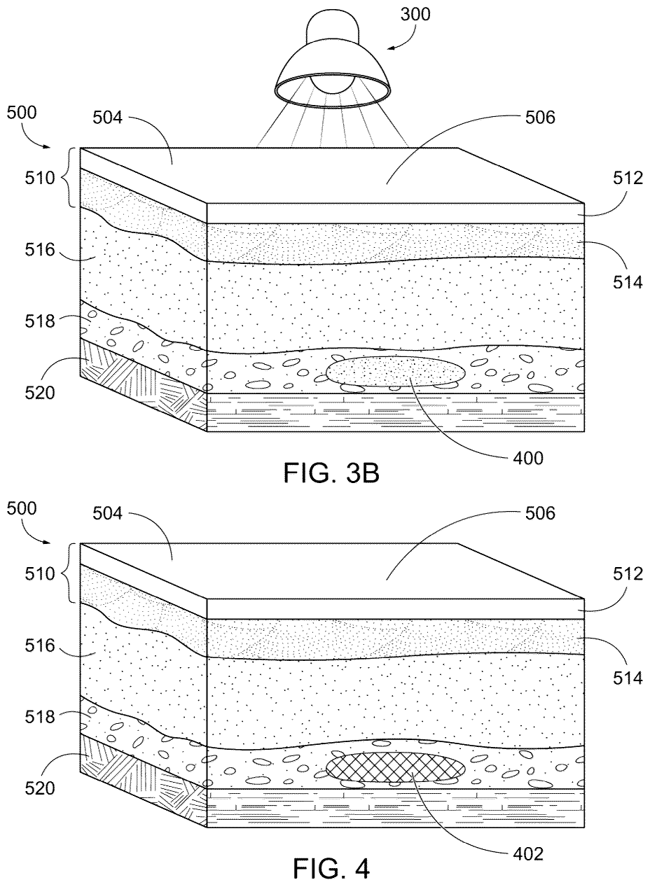

FIGS. 3A-4 illustrate one embodiment of a method of treating a depressed tissue portion. Depressed tissue portion 502 is shown as a concave portion of skin surface 504. The photocurable composition 400 is introduced beneath depressed tissue portion 502. In the illustrated embodiment a syringe 400, having needle 202 and distal needle tip 204 utilized to deliver photocurable composition 400 to a target region within subcutaneous tissue layer 518 located beneath depressed tissue portion 502. In accordance with certain embodiments, light source 300 is utilized to irradiate the photocurable composition through the overlying tissue layers. For example, the photocurable composition may be irradiated through the superficial epidermal tissue layer, the subsurface epidermal tissue layer, the dermis, the subcutaneous tissue layer, and/or the muscle tissue layer. As disclosed herein, such irradiation is sufficient to induce a photocrosslinking reaction forming crosslinks between individual polymers of the matrix forming polymer within the photocurable composition, thereby forming a crosslinked composition 402.

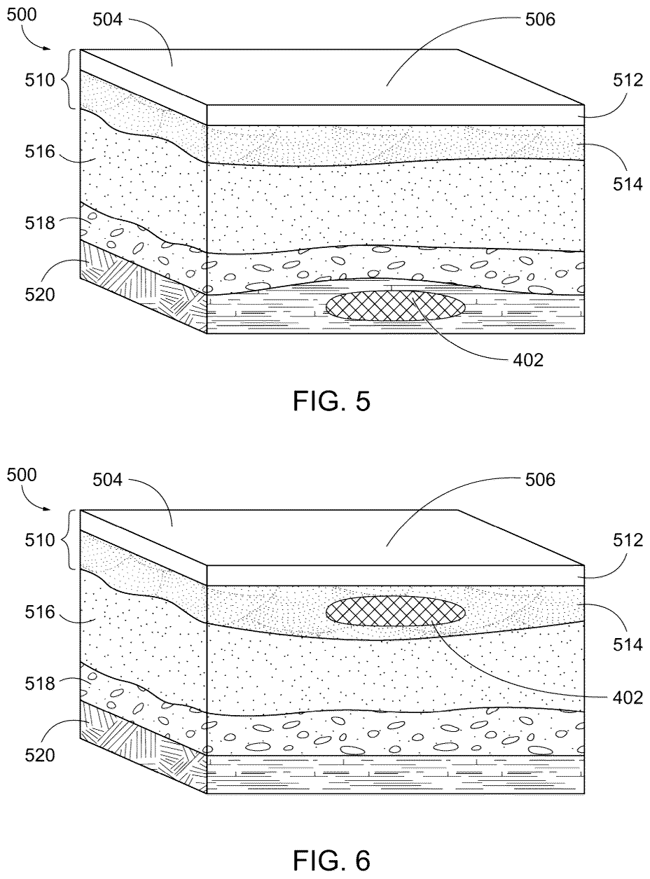

As disclosed herein, the photocurable composition may be introduced into any suitable layer beneath the depressed tissue portion. As shown in FIG. 4, a subcutaneous injection may be used to introduce the photocurable composition into a subcutaneous tissue layer. As shown in FIG. 5, an intramuscular injection may be used to introduce the photocurable composition into a intramuscular tissue location. As shown in FIG. 6, an intradermal injection may be used to introduce the photocurable composition into the epidermal tissue layer. It is also within the scope of the disclosure to provide a photocurable composition introduced into a location spanning two or more of the tissue layers.

In the illustrated embodiments, the soft tissue void is shown as existing within a single layer, however it is within the scope of the present disclosure to provide methods of filling a soft tissue void which spans between multiple tissue layers, or in other words a soft tissue void having void walls comprising two or more tissue layers. It is also within the scope of the present disclosure to introduce photocurable compositions as provided herein within locations spanning two or more adjacent tissue layers. For example, methods as disclosed herein may include injection a photocurable composition into the muscle and subcutaneous tissue layers, into the subcutaneous and dermis tissue layers, and/or into the epidermis and dermis tissue layers.

Particular advantage can be provided by including a remodelable collagenous material. Such remodelable collagenous materials can be provided, for example, by collagenous materials isolated from a suitable tissue source from a warm-blooded vertebrate, and especially a mammal. Reconstituted or naturally-derived collagenous materials can be used in the present invention. Such materials that are at least bioresorbable will provide advantage in the present invention, with materials that are bioerodable and promote cellular invasion and ingrowth providing particular advantage. Remodelable materials may be used in this context to promote cellular growth within the site in which a medical product of the invention is implanted. Moreover, the thickness of the medical product can be adjusted to control the extent of cellular ingrowth.

Suitable bioerodable materials can be provided by collagenous extracellular matrix materials (ECMs) possessing biotrophic properties, including in certain forms angiogenic collagenous extracellular matrix materials. For example, ECMs include materials such as submucosa, renal capsule membrane, dermal collagen, dura mater, pericardium, fascia lata, serosa, peritoneum or basement membrane layers, including liver basement membrane. Suitable submucosa-containing materials for these purposes include, for instance, materials that include intestinal submucosa, including small intestinal submucosa, stomach submucosa, urinary bladder submucosa, and uterine submucosa. These identified submucosa or other layers can occur in the ECM material alone, or in combination with other materials such as those derived from one or more adjacent layers in the source tissue.

The submucosa-containing ECM can be derived from any suitable organ or other biological structure, including for example submucosa derived from the alimentary, respiratory, intestinal, urinary or genital tracts of warm-blooded vertebrates. Submucosa-containing materials useful in the present invention can be obtained by harvesting such tissue sources and delaminating the submucosa (alone or combined with other materials) from smooth muscle layers, mucosal layers, and/or other layers occurring in the tissue source. For additional information as to submucosal materials useful in the present invention, and its isolation and treatment, reference can be made, for example, to U.S. Pat. Nos. 4,902,508, 5,554,389, 5,993,844, 6,206,931, and 6,099,567.

As prepared, the submucosal material and any other ECM used may optionally retain growth factors or other bioactive components native to the source tissue. For example, the submucosal or other ECM may include one or more native growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a native bioactive component that induces, directly or indirectly, a cellular response such as a change in cell morphology, proliferation, growth, protein or gene expression.

Submucosal or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive components, the ECM material can retain these components interspersed as solids between, upon and or within the collagen fibers. Particularly desirable naturally-derived ECM materials for use in the invention will include significant amounts of such interspersed, non-collagenous solids that are readily ascertainable under light microscopic examination. Such non-collagenous solids can constitute a significant percentage of the dry weight of the ECM material in certain inventive embodiments, for example at least about 1%, at least about 3%, and at least about 5% by weight in various embodiments of the invention.

Further, in addition or as an alternative to the inclusion of native bioactive components, non-native bioactive components such as those synthetically produced by recombinant technology or other methods, may be incorporated into the photocurable composition, submucosal or other ECM tissue. These non-native bioactive components may be naturally-derived or recombinantly produced proteins that correspond to those natively occurring in the ECM tissue, but perhaps of a different species (e.g. human proteins applied to collagenous ECMs from other animals, such as pigs). The non-native bioactive components may also be drug substances. Illustrative drug substances that may be incorporated into the photocurable compositions and/or ECM materials used in the invention include, for example, antibiotics, thrombus-promoting substances such as blood clotting factors, e.g. thrombin, fibrinogen, and the like. These substances may be applied to the ECM material as a premanufactured step, immediately prior to the procedure (e.g. by soaking the material in a solution containing a suitable antibiotic such as cefazolin), or during or after engraftment of the material on the patient. These substances may be added to the photocurable composition and/or a precursor of the photocurable composition. Alternatively, or additionally, a non-native bioactive component can be included in the coating material of the medical product. When included in the coating, the non-native bioactive component can be added at any point during preparation of the medical product, including being mixed with one or all of the coating components prior to application of the coating to a surface of a layer of a medical material or, alternatively, after the coating is formed, applied, or cross-linked.

A non-native bioactive component can be applied to a submucosal or other ECM tissue by any suitable means. Suitable means include, for example, spraying, impregnating, dipping, etc. The non-native bioactive component can be applied to the ECM tissue either before or after the coating is applied to the material, or both. Similarly, if other chemical or biological components are included in the ECM tissue, the non-native bioactive component can be applied either before, in conjunction with, or after these other components.

Submucosal or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosal or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosal tissue used in the present invention.

The present disclosure provides photocurable compositions providing advantageous physiochemical properties when they interact with patient tissue. Upon introduction into a patient tissue region and irradiation, crosslinks are formed between polymers of the matrix forming polymer within the photocurable composition as well as between such polymers and surrounding patient tissue. The photocurable compositions upon curing thus adhere patient tissue portions to one another through crosslinks between the matrix forming polymer and patient tissue as well as through crosslinks between molecules of the matrix forming polymer. In addition or alternatively, the photocurable compositions upon curing can promote the infiltration of patient tissue into cured volumes of the photocurable compositions. These physiochemical effects enhance other interactions with patient tissue as discussed herein, including for example in bulking of patient tissue. Thus, biological and/or chemical responses between patient tissue and the introduced photocurable and photocured compositions can enhance medical treatments as discussed herein.

An external light source as described herein may be any light source suitable for applying visible light to the skin or tissue graft and thereby photocrosslinking the photocurable composition from outside of the body. For example, a broad-spectrum white LED or incandescent light source may be utilized as a light source. In particular, any light source capable of light in the blue spectrum, i.e. having wavelengths of about 450 nm to about 485 nm may be utilized as a light source. Other suitable light sources may be used so long as crosslinking occurs within the photocurable composition within the body. Light is applied at an intensity, and for a duration sufficient to cause crosslinking within the photocurable composition. The light source can be situated above the skin surface or directly on the skin surface. In accordance with some forms, the photocurable composition is irradiated (e.g. the external light source is applied) for five seconds to five minutes, preferably ten seconds to four minutes, even more preferably for twenty seconds to three minutes. In accordance with certain embodiments, the photocurable composition is irradiated for about thirty seconds. In accordance with certain embodiments, the photocurable composition is irradiated for about sixty seconds. In accordance with certain embodiments, the photocurable composition is irradiated for about two minutes. In accordance with certain embodiments, the photocurable composition is irradiated for about three minutes. In accordance with some forms, the external light source is applied directly on the external tissue (e.g. skin surface) overlying the injected photocurable composition. In certain embodiments the external light source is spaced a distance from the external tissue surface. For example, in some forms the external light source is applied at a distance of zero to ten centimeters from the external tissue surface, preferably 0.5 to 5 centimeters. In certain embodiments, the external light source is spaced about one centimeter from the external tissue surface.

Any or all of the components described herein can be provided in a sterile package for providing necessary parts, or a variety of parts, to a surgeon. For example, one or more preloaded syringes containing a photocurable composition precursor and/or an activator may be provided in a single sterile package or kit. In some forms the photocurable composition precursor and/or activator may be provided in separate sterile packages. Sterilization may be achieved, for example, by irradiation, ethylene oxide gas, or any other suitable sterilization technique, and the materials and other properties of the medical packaging will be selected accordingly. In accordance with some forms, the medical packaging selected may be opaque to prevent additional exposure of the medical graft material to light. Alternatively, sterile kits containing predetermined sizes or types of components may be provided. Packages or kits of the components described herein can include additional devices or tools which may be useful in the particular medical procedure being performed.

To promote a further understanding of embodiments disclosed herein and their features and advantages, the following specific Examples are provided. It will be understood that these examples are illustrative and not limiting in nature.

### EXAMPLE 1

### Preparation of Photocurable Composition Precursor

A photocurable composition precursor was prepared comprising: 27.47% w/v unmodified gelatin, 4.107 M urea, 0.01 M phosphate buffered saline, and 0.756 mM tris(2,2′-bipyridyl) ruthenium (II) chloride hexahydrate.

### EXAMPLE 2

Subdermal and Intramuscular Injection and Curing of Photocurable Composition 0.9 mL of the photocurable composition precursor as described in Example 1 was loaded into a 1 mL syringe. The photocurable composition was prepared by mixing the precursor with 0.1 mL 1 M sodium persulfate, prepared fresh, using a luer-to-luer connector. A 25-gauge hypodermic needle marked at ⅛′ intervals was affixed to the syringe containing the photocurable composition. For this experiment, skin-on chicken breast was used as an injection substrate. The mixed photocurable composition was injected into the chicken breast at fixed injection depths using the marking on the needle. For subdermal injections, 0.4 mL of the photocurable composition was injected. For intramuscular injections, 0.2 mL of the photocurable composition was injected. An LED operating lamp was used at various light intensities and various lamp heights to cure the injected material through the overlying tissues. Table 1 details the results. A lamp height of 0 cm indicates that the light source was placed directly on the tissue surface. For longer cure times (sample 6 and 7), the light source was slowly moved across the tissue surface in a 1 cm) area around the injection sire during curing. For all other groups, the lamp was held still at or above the injection location during curing.

| | Injection | Cure | Lamp | Lamp | | Cure |
|---|---|---|---|---|---|---|
| | Depth | time | Height | Intensity | | Depth |
| Sample # | (inches) | (s) | (cm) | (%) | Cured | (mm) |
| 1 | Subdermal | 30 | 1 | 70 | Y | Subdermal |
| | (~1/16) | | | | | (~1/16″) |
| 2 | 1/4 | 30 | 1 | 100 | N | N/A |
| 3 | 1/8 | 30 | 1 | 100 | Y | 10 |
| 4 | 1/4 | 30 | 1 | 100 | Partial | Not |
| | | | | | | measured |
| 5 | 1/4 | 60 | 1 | 100 | Partial | Subdermal |
| | | | | | | (~1/16*) |
| 6 | 1/4 | 120 | 0 | 100 | Y | 10 |
| 7 | 1/2 | 180 | 0 | 100 | Y | 25 |

These results show that the photocurable compositions described herein can be injected subdermally and intramuscularly and cured using an operating lamp. Deeper injections require higher lamp intensity, longer cure time, and/or closer lamp height to the tissue surface. Cure depths of up to 30 mm were achieved in this study, deeper cure depths may be achievable.

### EXAMPLE 3

Phenol Enriched Gelatin Prepared with Bolton Hunter Reagent

Nippi MediGelatin (derived from porcine skin; Mw approximately 100 kilodaltons) was dissolved in high purity water with 6.18 g/L boric acid, 9.54 g/L sodium borate, and 4.38 g/L sodium chloride at 30° C. and 60° C. at a concentration of 10 g/L in a 1 L reaction volume (n=1). 200 mL aliquots of this solution were extracted into 500 mL Erlenmeyer flasks and combined with 3.5 mL of a Bolton Hunter reagent/DMSO solution (Bolton Hunter reagent N-succinimidyl-3-[4-hydroxyphenyl]propionate). The Bolton Hunter/DMSO solution was prepared at a concentration such that the final concentration of Bolton Hunter reagent in the gelatin solution ranged from 0.2 to 5 g/L. The mixture was reacted at 40° C. in a shaken incubator for two hours. The solution was dialyzed, dried, and analyzed for phenol content using absorbance at 280 nm. Unless noted otherwise, groups had a replicate size of n=3.

Each group was evaluated for normality and compared across groups for equal variance. Groups were then compared for statistical difference using a one-way ANOVA (α=0.05) and Tukey post-hoc tests.

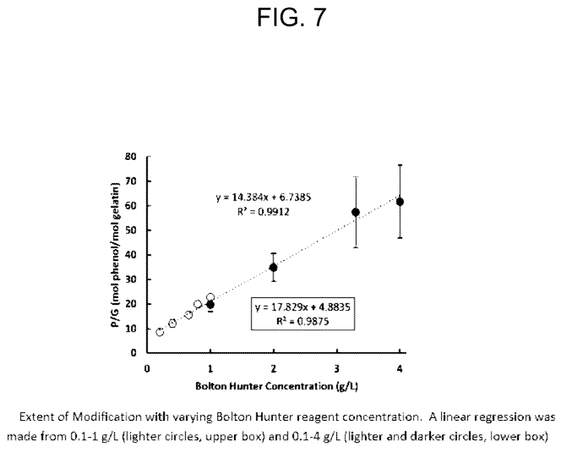

The results are summarized in the FIG. 7. P/G values (mole phenol/mole gelatin) for the modified materials ranged from just under 10 to about 60 (the unmodified gelatin had a P/G value of about 3). At all concentrations at and below 1 g/L Bolton Hunter, the resulting phenol enriched gelatin was soluble in PBS. The relationship between Bolton Hunter concentration and extent of phenol enrichment was linear, with a 10% change in Bolton Hunter concentration causing a ˜6% change in the molar ratio of phenol to gelatin. At Bolton Hunter concentrations at 2 g/L and higher, the relationship became less linear and the measurements had higher standard deviation. In addition, the modified gelatin became less soluble in PBS, with protein modified at 5 g/L becoming completely insoluble. These results indicate that a severe excess of Bolton Hunter reagent and a resulting very high P/G value for the phenol enriched protein can decrease the solubility of the modified protein in an aqueous medium.

### EXAMPLE 4

Phenol Enriched Gelatin Prepared by Carbodiimide Reaction Gelatin was modified to include additional phenol groups using EDC (l-ethyl-3-(3-dimethylaminopropyl)carbodiimide hydrochloride), NHS (N-hydroxysuccinimide), and HPPA (3-(4-Hydroxyphenyl)propionic acid). A precipitate was first prepared using a 5:2:1 ratio of NHS:HDC:HPPA at concentrations of 325 mM NHS, 130 mM EDC and 65 mM HPPA. First, HPPA was solubilized in a 0.1M MES, 0.9% Sodium Chloride, pH 4.7 buffer on a stir plate at 200 rpm. Once the HPPA was dissolved, the EDC and MIS were added to the solution. After 15-20 minutes a precipitate began to form. The solution was allowed to react for 2 to 4 hours and then vacuum filtered. Following double filtration of the solution, the precipitate captured on the filter paper was allowed to dry in a fume hood for at least 24 hours. Once the precipitate was dry, it was utilized as solubilized in DMSO in 4× mass in place of the Bolton-Hunter Reagent to add phenolic groups to the gelatin.

Modified gelatin prepared using the precipitate in place of Bolton-Hunter reagent was formulated into photocurable adhesive compositions using a phosphate buffered saline medium, bipyridyl) ruthenium (II) chloride hexahydrate and sodium persulfate. Photocurable adhesive formulations of having 5:1, 1:1 and 0:1 ratios of unmodified gelatin to modified gelatin were prepared. The thus prepared photocurable adhesive demonstrated the ability to cure under visible light.

The use of the terms “a” and “an” and “the” and similar referents in the context of describing the invention especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EMBODIMENTS

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.
1. A method of treating diseased or damaged tissue in a patient, the method comprising:
   introducing a photocurable composition to the diseased or damaged tissue, the photocurable composition comprising a photoactivatable metal ligand complex, a matrix forming polymer, and an electron acceptor, and wherein the photocurable composition is positioned behind a tissue portion; and irradiating the photocurable composition through the tissue portion to induce a photocrosslinking reaction forming crosslinks between individual polymers of the matrix forming polymer within the photocurable composition.
2. The method of embodiment 1, wherein the tissue portion comprises intact patient tissue superficial to the diseased or damaged tissue.
3. The method of any one of embodiments 1 or 2, wherein said introducing comprises injecting the photocurable composition into a subdermal space.
4. The method of any one of embodiments 1 or 2, wherein said introducing comprises injecting the photocurable composition into an intramuscular space.
5. The method of embodiment 1, wherein the tissue portion comprises a tissue graft applied over the photocurable composition.
6. The method of any one of embodiments 1 to 5, wherein the tissue portion has a thickness of about 1 mm to about 50 mm.
7. The method of any one of embodiments 1 to 6, wherein the photoactivatable metal ligand complex comprises a Ru(II) bipyridyl complex.
8. The method of any one of embodiments 1 to 7, wherein the matrix forming polymer comprises a matrix protein.
9. The method of embodiment 8, wherein the matrix protein comprises gelatin.
10. The method of any one of embodiments 1 to 9, wherein the matrix forming polymer comprises a synthetic polymer.
11. The method of any one of embodiments 1 to 10, wherein the electron acceptor comprises sodium persulfate.
12. The method of any one of embodiments 1 to 11, wherein said irradiating is performed with an external light source.
13. The method of embodiment 12, wherein said external light source is configured to emit visible light.
14. The method of any one of embodiments 1 to 13, wherein said irradiating is performed for at least 30 seconds.
15. The method of any one of embodiments 1 to 14, wherein said irradiating is performed to induce a photocrosslinking reaction forming crosslinks between individual polymers of the matrix forming polymer within the photocurable composition, and crosslinks between polymers of the matrix forming polymer and patient tissue.
16. A method of treating a depressed tissue portion in a patient, the method comprising:
   injecting a photocurable composition beneath the depressed tissue portion, the photocurable composition comprising a photoactivatable metal ligand complex, a matrix forming polymer, and an electron acceptor; and
   irradiating the photocurable composition through the patient's skin to induce a photocrosslinking reaction forming crosslinks between individual polymers of the matrix forming polymer within the photocurable composition.
17. The method of embodiment 16, wherein said injecting comprises injecting the photocurable composition into a subdermal space.
18. The method of any one of embodiments 16 or 17, wherein said injecting comprises injecting the photocurable composition into an intramuscular space.
19. The method of any one of embodiments 16 to 18, wherein the photocurable composition is injected in a target area 1 mm to about 50 mm beneath the surface of the patient's skin.
20. The method of any one of embodiments 16 to 19, wherein the photoactivatable metal ligand complex comprises a Ru(II) bipyridyl complex.
21. The method of any one of embodiments 16 to 20, wherein the matrix forming polymer comprises a matrix protein.
22. The method of embodiment 21, wherein the matrix protein comprises gelatin.
23. The method of any one of embodiments 16 to 22, wherein the matrix forming polymer comprises a synthetic polymer.
24. The method of any one of embodiments 16 to 23, wherein the electron acceptor comprises sodium persulfate.
25. The method of any one of embodiments 16 to 24, wherein said irradiating is performed with an external light source.
26. The method of embodiment 25, wherein said external light source is configured to emit visible light.
27. The method of any one of embodiments 16 to 26, wherein said irradiating is performed for at least 30 seconds.
28. The method of any one of embodiments 16 to 27, wherein said irradiating is performed to induce a photocrosslinking reaction forming crosslinks between individual polymers of the matrix forming polymer within the photocurable composition, and crosslinks between polymers of the matrix forming polymer and patient tissue.
29. A medical kit comprising:
   a sterile package;
   a photocurable composition contained within said sterile package, the photocurable composition comprising a photoactivatable metal ligand complex, a matrix forming polymer, and an electron acceptor, and
   a syringe configured to inject said photocurable composition into a subdermal or intramuscular location of a patient.
30. The medical kit of embodiment 29, also comprising a light source configured to induce a photocrosslinking reaction within the photocurable composition upon exposure to irradiation from the light source through patient tissue.
31. The medical kit of any one of embodiments 29 or 30, wherein said photocurable composition is contained within said syringe.
32. A method of treating an open cutaneous wound in a patient, the method comprising:
   applying a photocurable composition to the open cutaneous wound, the photocurable composition comprising a photoactivatable metal ligand complex, a matrix forming polymer, and an electron acceptor;
   applying a tissue graft over the open cutaneous wound; and
   irradiating the photocurable composition through the tissue graft to induce a photocrosslinking reaction forming crosslinks between individual polymers of the matrix forming polymer within the photocurable composition.
33. The method of embodiment 32, wherein the tissue graft has a thickness of about 1 mm to about 50 mm.
34. The method of one of embodiments 32 or 33, wherein the photoactivatable metal ligand complex comprises a Ru(II) bipyridyl complex.
35. The method of any one of embodiments 32 to 34, wherein the matrix forming polymer comprises a matrix protein.
36. The method of embodiment 35, wherein the matrix protein comprises gelatin.
37. The method of any one of embodiments 32 to 36, wherein the matrix forming polymer comprises a synthetic polymer.
38. The method of any one of embodiments 32 to 37, wherein the electron acceptor comprises sodium persulfate.
39. The method of any one of embodiments 32 to 38, wherein said irradiating is performed with an external light source configured to emit visible light.
40. The method of any one of embodiments 32 to 39, wherein said irradiating is performed for at least 30 seconds.
41. The method of any one of embodiments 32 to 40, wherein said irradiating is performed to induce a photocrosslinking reaction forming crosslinks between individual polymers of the matrix forming polymer within the photocurable composition, and crosslinks between polymers of the matrix forming polymer and tissue graft.
42. The medical kit of any one of embodiments 29 to 31, wherein the syringe comprises a cannulated needle configured to pierce patient tissue.
43. The method of any one of embodiments 1 to 15, wherein said introducing comprises injecting the photocurable composition through a needle or other cannulated device.
44. The method of any one of embodiments 16 to 28, wherein said injecting comprises injecting the photocurable composition through a needle or other cannulated device.
45. A photocurable composition comprising a photoactivatable metal ligand complex, a matrix forming polymer, and an electron acceptor, for use in treating diseased or damaged tissue in a patient by a method comprising:
   introducing the photocurable composition to the diseased or damaged tissue, and wherein the photocurable composition is positioned behind a tissue portion; and
   irradiating the photocurable composition through the tissue portion to induce a photocrosslinking reaction forming crosslinks between individual polymers of the matrix forming polymer within the photocurable composition.
46. The photocurable composition of embodiment 45, wherein the tissue portion comprises intact patient tissue superficial to the diseased or damaged tissue.
47. The photocurable composition of any one of embodiments 45 or 46, wherein said introducing comprises injecting the photocurable composition into a subdermal space.
48. The photocurable composition of any one of embodiments 45 or 46, wherein said introducing comprises injecting the photocurable composition into an intramuscular space.
49. The photocurable composition of embodiment 45, wherein the tissue portion comprises a tissue graft applied over the photocurable composition.
50. The photocurable composition of any one of embodiments 45 to 49, wherein the tissue portion has a thickness of about 1 mm to about 50 mm.
51. The photocurable composition of any one of embodiments 45 to 50, wherein the photoactivatable metal ligand complex comprises a Ru(II) bipyridyl complex.
52. The photocurable composition of any one of embodiments 45 to 51, wherein the matrix forming polymer comprises a matrix protein.
53. The photocurable composition of embodiment 52, wherein the matrix protein comprises gelatin.
54. The photocurable composition of any one of embodiments 45 to 53, wherein the matrix forming polymer comprises a synthetic polymer.
55. The photocurable composition of any one of embodiments 45 to 54, wherein the electron acceptor comprises sodium persulfate.
56. The photocurable composition of any one of embodiments 45 to 55, wherein said irradiating is performed with an external light source.
57. The photocurable composition of embodiment 56, wherein said external light source is configured to emit visible light.
58. The photocurable composition of any one of embodiments 45 to 57, wherein said irradiating is performed for at least 30 seconds.
59. The photocurable composition of any one of embodiments 45 to 58, wherein said irradiating is performed to induce a photocrosslinking reaction forming crosslinks between individual polymers of the matrix forming polymer within the photocurable composition, and crosslinks between polymers of the matrix forming polymer and patient tissue.
60. A photocurable composition comprising a photoactivatable metal ligand complex, a matrix forming polymer, and an electron acceptor, for use in treating a depressed tissue portion in a patient by a method comprising:
   injecting the photocurable composition beneath the depressed tissue portion; and
   irradiating the photocurable composition through the patient's skin to induce a photocrosslinking reaction forming crosslinks between individual polymers of the matrix forming polymer within the photocurable composition.
61. The photocurable composition of embodiment 60, wherein said injecting comprises injecting the photocurable composition into a subdermal space.
62. The photocurable composition of embodiment 60, wherein said injecting comprises injecting the photocurable composition into an intramuscular space.
63. The photocurable composition of any one of embodiments 60 to 62, wherein the photocurable composition is injected in a target area 1 mm to about 50 mm beneath the surface of the patient's skin.
64. The photocurable composition of any one of embodiments 60 to 63, wherein the photoactivatable metal ligand complex comprises a Ru(II) bipyridyl complex.
65. The photocurable composition of any one of embodiments 60 to 64, wherein the matrix forming polymer comprises a matrix protein.
66. The photocurable composition of embodiment 65, wherein the matrix protein comprises gelatin.
67. The photocurable composition of any one of embodiments 60 to 66, wherein the matrix forming polymer comprises a synthetic polymer.
68. The photocurable composition of any one of embodiments 60 to 67, wherein the electron acceptor comprises sodium persulfate.
69. The photocurable composition of any one of embodiments 60 to 68, wherein said irradiating is performed with an external light source.
70. The photocurable composition of embodiment 69, wherein said external light source is configured to emit visible light.
71. The photocurable composition of any one of embodiments 60 to 70, wherein said irradiating is performed for at least 30 seconds.
72. The photocurable composition of any one of embodiments 60 to 71, wherein said irradiating is performed to induce a photocrosslinking reaction forming crosslinks between individual polymers of the matrix forming polymer within the photocurable composition, and crosslinks between polymers of the matrix forming polymer and patient tissue.
73. The photocurable composition of any one of embodiments 45 to 59, wherein the diseased or damaged tissue is sphincter tissue and/or wherein the treating diseased or damaged tissue is to treat fecal incontinence or urinary incontinence in the patient.
74. The photocurable composition of any one of embodiments 45 to 59, wherein the diseased or damaged tissue is laryngeal tissue and/or wherein the treating is to modify or support the vocal folds.
75. The photocurable composition of any one of embodiments 45 to 59, or claim 74, wherein the treating is to treat: lesions, polyps, nodules, and/or cysts on the vocal fold, and/or voids left after removal of vocal fold lesions, polyps, nodules, and cysts; scarred vocal folds; and/or paralysis of vocal folds.
76. The photocurable composition of any one of embodiments 60 to 72, wherein the treating a depressed tissue portion is to treat a scar, optionally wherein the scar is an acne scar.
77. A photocurable composition comprising a photoactivatable metal ligand complex, a matrix forming polymer, and an electron acceptor, for use in treating an open cutaneous wound in a patient by a method comprising:
   applying the photocurable composition to the open cutaneous wound;
   applying a tissue graft over the open cutaneous wound; and
   irradiating the photocurable composition through the tissue graft to induce a photocrosslinking reaction forming crosslinks between individual polymers of the matrix forming polymer within the photocurable composition.
78. The method of embodiment 77, wherein the tissue graft has a thickness of about 1 mm to about 50 mm.
79. The method of any one of embodiments 77 or 78, wherein the photoactivatable metal ligand complex comprises a Ru(II) bipyridyl complex.
80. The method of any one of embodiments 77 to 79, wherein the matrix forming polymer comprises a matrix protein.
81. The method of embodiment 80, wherein the matrix protein comprises gelatin.
82. The method of any one of embodiments 77 to 81, wherein the matrix forming polymer comprises a synthetic polymer.
83. The method of any one of embodiments 77 to 82, wherein the electron acceptor comprises sodium persulfate.
84. The method of any one of embodiments 77 to 83, wherein said irradiating is performed with an external light source configured to emit visible light.
85. The method of any one of embodiments 77 to 84, wherein said irradiating is performed for at least 30 seconds.
86. The method of any one of embodiments 77 to 85, wherein said irradiating is performed to induce a photocrosslinking reaction forming crosslinks between individual polymers of the matrix forming polymer within the photocurable composition, and crosslinks between polymers of the matrix forming polymer and tissue graft.
87. A photocurable composition comprising a photoactivatable metal ligand complex, a matrix forming polymer, and an electron acceptor, for use in treating:
   (a) fecal incontinence or urinary incontinence in a patient; or
   (b) lesions, polyps, nodules, and/or cysts on a vocal fold, and/or voids left after removal of vocal fold lesions, polyps, nodules, and cysts; scarred vocal folds; and/or paralysis of vocal folds, in a patient; or
   (c) a depressed scar in a patient, optionally wherein the depressed scar is an acne scar; or
   (d) an open cutaneous wound in a patient.
88. The photocurable composition of embodiment 87, for use in treating fecal incontinence or urinary incontinence in a patient.
89. The photocurable composition of embodiment 88, for use in treating fecal incontinence or urinary incontinence in a patient by a method comprising:
   introducing the photocurable composition into or near a sphincter; and
   irradiating the photocurable composition to induce a photocrosslinking reaction forming crosslinks between individual polymers of the matrix forming polymer within the photocurable composition.
90. The photocurable composition of embodiment 89, wherein the introduced photocurable composition is behind a tissue portion, and wherein the irradiating is through the tissue portion.
91. The photocurable composition of embodiment 87, for use in treating lesions, polyps, nodules, and or cysts on a vocal fold, and/or voids left after removal of vocal fold lesions, polyps, nodules, and cysts; scarred vocal folds; and/or paralysis of vocal folds, in a patient.
92. The photocurable composition of embodiment 91, wherein the treating is by a method comprising:
   to introducing the photocurable composition into laryngeal tissue of the patient; and
   irradiating the photocurable composition to induce a photocrosslinking reaction forming crosslinks between individual polymers of the matrix forming polymer within the photocurable composition.
93. The photocurable composition of embodiment 87, for use in treating a depressed scar in a patient.
94. The photocurable composition of embodiment 93, wherein said treating is by a method comprising:
   introducing the photocurable composition beneath the depressed scar; and
   irradiating the photocurable composition to induce a photocrosslinking reaction forming crosslinks between individual polymers of the matrix forming polymer within the photocurable composition.
95. The photocurable composition of embodiment 94, wherein the introduced photocurable composition is behind a tissue portion, and wherein the irradiating is through the tissue portion.
96. The photocurable composition of any one of embodiments 93 to 95, wherein the depressed scar is an acne scar.
97. The photocurable composition of embodiment 87, for treating an open cutaneous wound in a patient.
98. The method of embodiment 97, wherein said treating is by a method comprising:
   applying the photocurable composition to the open cutaneous wound;
   applying a tissue graft over the open cutaneous wound; and
   irradiating the photocurable composition to induce a photocrosslinking reaction forming crosslinks between individual polymers of the matrix forming polymer within the photocurable composition.
99. The photocurable composition of embodiment 98, wherein said irradiating is through the tissue graft.
100. The photocurable composition of any one of embodiments 87 to 99, wherein the photoactivatable metal ligand complex comprises a Ru(II) bipyridyl complex.
101. The photocurable composition of any one of embodiments 87 to 100, wherein the matrix forming polymer comprises a matrix protein.
102. The photocurable composition of embodiment 101, wherein the matrix protein comprises gelatin.
103. The photocurable composition of any one of embodiments 87 to 100, wherein the matrix forming polymer comprises a synthetic polymer.
104. The photocurable composition of any one of embodiments 87 to 103, wherein the electron acceptor comprises sodium persulfate.
105. The photocurable composition of any one of embodiments 89, 90, 92, 94, 95, 98, or 99, wherein said irradiating is performed with an external light source.
106. The photocurable composition of embodiment 105, wherein said external light source is configured to emit visible light.
107. The photocurable composition of any one of embodiments 89, 90, 92, 94, 95, 98, or 99, wherein said irradiating is performed for at least 30 seconds.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A method of treating diseased or damaged tissue in a patient, the method comprising:
introducing a photocurable composition to the diseased or damaged tissue, the photocurable composition comprising a photoactivatable metal ligand complex, a matrix forming polymer, and an electron acceptor, and wherein the photocurable composition is positioned behind a tissue portion; and
irradiating the photocurable composition through the tissue portion to induce a photocrosslinking reaction forming crosslinks between individual polymers of the matrix forming polymer within the photocurable composition.

2. The method of claim 1, wherein the tissue portion comprises intact patient tissue superficial to the diseased or damaged tissue, or a tissue graft applied over the photocurable composition.

3. The method of claim 2, wherein said introducing comprises injecting the photocurable composition into a subdermal space and/or an intramuscular space.

4-5. (canceled)

6. The method of claim 1, wherein the tissue portion has a thickness of about 1 mm to about 50 mm.

7. (canceled)

8. The method of claim 1, wherein the matrix forming polymer comprises a matrix protein, preferably gelatin.

9. (canceled)

10. The method of claim 1, wherein the matrix forming polymer comprises a synthetic polymer.

11. (canceled)

12. The method of claim 1, wherein said irradiating is performed with an external light source, preferably wherein said external light source is configured to emit visible light.

13-14. (canceled)

15. The method of claim 1, wherein said irradiating is performed to induce a photocrosslinking reaction forming crosslinks between individual polymers of the matrix forming polymer within the photocurable composition, and crosslinks between polymers of the matrix forming polymer and patient tissue.

16. A method of treating a depressed tissue portion in a patient, the method comprising:
injecting a photocurable composition beneath the depressed tissue portion, the photocurable composition comprising a photoactivatable metal ligand complex, a matrix forming polymer, and an electron acceptor; and
irradiating the photocurable composition through the patient's skin to induce a photocrosslinking reaction forming crosslinks between individual polymers of the matrix forming polymer within the photocurable composition.

17. The method of claim 16, wherein said injecting comprises injecting the photocurable composition into a subdermal space and/or intramuscular space.

18. (canceled)

19. The method of claim 16, wherein the photocurable composition is injected in a target area 1 mm to about 50 mm beneath the surface of the patient's skin.

20. (canceled)

21. The method of claim 16, wherein the matrix forming polymer comprises a matrix protein, preferably gelatin.

22. (canceled)

23. The method of claim 16, wherein the matrix forming polymer comprises a synthetic polymer.

24. (canceled)

25. The method of claim 16, wherein said irradiating is performed with an external light source, preferably wherein said external light source is configured to emit visible light.

26-27. (canceled)

28. The method of claim 16, wherein said irradiating is performed to induce a photocrosslinking reaction forming crosslinks between individual polymers of the matrix forming polymer within the photocurable composition, and crosslinks between polymers of the matrix forming polymer and patient tissue.

29-86. (canceled)

87. A photocurable composition comprising a photoactivatable metal ligand complex, a matrix forming polymer, and an electron acceptor, for use in treating:
(a) fecal incontinence or urinary incontinence in a patient; or
(b) lesions, polyps, nodules, and/or cysts on a vocal fold, and/or voids left after removal of vocal fold lesions, polyps, nodules, and cysts; scarred vocal folds; and/or paralysis of vocal folds, in a patient; or
(c) a depressed scar in a patient, optionally wherein the depressed scar is an acne scar; or
(d) an open cutaneous wound in a patient.

88. The photocurable composition of claim 87, for use in treating fecal incontinence or urinary incontinence in a patient.

89. The photocurable composition of claim 88, for use in treating fecal incontinence or urinary incontinence in a patient by a method comprising:
introducing the photocurable composition into or near a sphincter; and
irradiating the photocurable composition to induce a photocrosslinking reaction forming crosslinks between individual polymers of the matrix forming polymer within the photocurable composition.

90. The photocurable composition of claim 89, wherein the introduced photocurable composition is behind a tissue portion, and wherein the irradiating is through the tissue portion.

91. The photocurable composition of claim 87, for use in treating lesions, polyps, nodules, and/or cysts on a vocal fold, and/or voids left after removal of vocal fold lesions, polyps, nodules, and cysts; scarred vocal folds; and/or paralysis of vocal folds, in a patient.

92. The photocurable composition of claim 91, wherein the treating is by a method comprising:
introducing the photocurable composition into laryngeal tissue of the patient; and
irradiating the photocurable composition to induce a photocrosslinking reaction forming crosslinks between individual polymers of the matrix forming polymer within the photocurable composition.

93. The photocurable composition of claim 87, for use in treating a depressed scar in a patient.

94. The photocurable composition of claim 93, wherein said treating is by a method comprising:
introducing the photocurable composition beneath the depressed scar; and
irradiating the photocurable composition to induce a photocrosslinking reaction forming crosslinks between individual polymers of the matrix forming polymer within the photocurable composition.

95. The photocurable composition of claim 94, wherein the introduced photocurable composition is behind a tissue portion, and wherein the irradiating is through the tissue portion.

96. The photocurable composition of claim 93, wherein the depressed scar is an acne scar.

97. The photocurable composition of claim 87, for treating an open cutaneous wound in a patient.

98. The method of claim 97, wherein said treating is by a method comprising:
applying the photocurable composition to the open cutaneous wound;
applying a tissue graft over the open cutaneous wound; and
irradiating the photocurable composition to induce a photocrosslinking reaction forming crosslinks between individual polymers of the matrix forming polymer within the photocurable composition.

99. The photocurable composition of claim 98, wherein said irradiating is through the tissue graft.

100. (canceled)

101. The photocurable composition of claim 87, wherein the matrix forming polymer comprises a matrix protein, preferably gelatin.

102. (canceled)

103. The photocurable composition of claim 87, wherein the matrix forming polymer comprises a synthetic polymer.

104-107. (canceled)
